(19) United States
(12) Patent Application Publication
(10) Pub. No.: US 2021/0039893 A1
(43) Pub. Date: Feb. 11, 2021

(54) A SYSTEM FOR TRANSPORTING BIOMASS MATERIAL AND A METHOD FOR PREVENTING BLOW BACK IN SAID SYSTEM

(71) Applicant: VALMET AB, Sundsvall (SE)

(72) Inventors: Patrik PETTERSSON, Alnö (SE); Johan CARLSSON, Alnö (SE); Stefan MELLANDER, Alnö (SE)

(73) Assignee: VALMET AB, Sundsvall (SE)

(21) Appl. No.: 16/967,769

(22) PCT Filed: Dec. 11, 2018
(86) PCT No.: PCT/SE2018/051288

§ 371 Date: Aug. 6, 2020

(30) Foreign Application Priority Data
Feb. 9, 2018 (SE) 1850144-5

(51) Int. Cl. B65G 43/02; D21C 7/06; B65G 33/22; B30B 9/12

(52) U.S. Cl. CPC B65G 43/02; D21C 7/06; B65G 33/22; B65G 2203/045; B65G 2203/0208; B65G 2203/042; B65G 2201/04; B30B 9/125

(57) ABSTRACT

The invention relates to a system (1) comprising a feeding device (2) comprising a channel (6) having an inlet (8) and an outlet (10) and a feed screw (12) for conveying biomass material through the channel. The feed screw comprises a screw flight (12b) that extends from a first end (12c) to a second end (12d) and is adapted to form a gas impermeable plug of biomass. The system comprises at least one primary measuring unit (14; 16) adapted to continuously measure a primary variable indicative of the gas permeability of the plug, which primary measuring unit is connected to said feeding device between the first end of the screw flight and the outlet; and a control unit (3) adapted to use said primary variable values to monitor the gas permeability of the plug. The invention also relates to a method for preventing blow back in the above described system.

### TECHNICAL FIELD

The invention relates to a system for transporting biomass material and a method for preventing blow back in said system.

### BACKGROUND

It is known to use feeding devices comprising one or more force feeding screws for conveying biomass from one part of a pulping process to another part of said pulping process. These devices may also be used for dewatering of the biomass during transport. When biomass is transported to a pressurized zone, the force feeding screws are suitably adapted to compress said biomass during transport to form a gas impermeable plug of biomass within the feeding device to prevent gas from the pressurized zone from flowing back against the biomass transport direction through the force feeding screw, so-called blow-back. Such feeding devices are referred to as plug screw feeders.

It is also known to arrange a blow back damper at a plug screw feeder outlet, which blow back damper is adapted to apply a counter pressure on the biomass discharged through said outlet to facilitate formation of the plug. The blow back damper may also be used to seal the opening when blow back occurs.

The risk of blow back occurring is always a concern and depends on a plurality of variables, e.g. the amount of biomass material continuously fed into the plug screw feeder, the material properties of said biomass material, the rotational speed of the feed screw, the counter-pressure applied to said biomass material by the blow back damper, the dimensions of the channel within the plug screw feeder and the pressure within the pressurized zone. To complicate things further, some of the above variables may vary during use, e.g. the pressure within the pressurized zone. Consequently, choosing some of these variables is a complex and difficult procedure.

Several prior art documents deal with the problem of blow-back.

U.S. Pat. No. 7,976,259 relates to a system comprising a plug screw feeder for feeding biomass into a pressurized vessel. A pressure sensor is adapted to monitor the air pressure within an inlet for biomass in a screw feeding housing, and an actuator connected to a compression disk and a drive mechanism connected to a first conveyor screw are adjusted in response to said measurements. When blow-back is detected by the pressure sensor, the actuator and drive mechanism are adjusted to effectuate an effectively sealed plug. U.S. Pat. No. 4,274,786 discloses a similar solution, wherein a counter pressure applied to a plate arranged at the outlet of a screw passage is regulated in response to steam pressure values received from a pressure transducer adapted to continuously measure the steam pressure in an inlet to the screw passage.

U.S. Pat. No. 3,756,434 relates to an alternative solution and discloses an apparatus for conveying bulk material, wherein a pressure measuring device is arranged to measure the pressure within a bunker arranged at an outlet of a delivery pipe. If the pressure in the bunker either increases or decreases above or below certain predetermined limits, the fluctuations are sensed by the pressure monitoring device and a signal is transmitted to an adjusting drive mechanism adapted to control a conveying worm within the delivery pipe.

A different solution is disclosed in US 2011/0271649 A1, wherein a primary and a secondary screw are arranged in series within a screw pipe and separated by an intermediate pipe section, which does not contain any conveying elements. An almost gas-tight plug of biomass is formed in the intermediate pipe section. The revolution speed of the primary screw conveyor largely determines the conveying capacity and the revolution speed of the secondary screw conveyor largely determines the sealing tightness of the plug. A pressure sensor is arranged within the intermediate pipe section to measure the gas pressure within the screw pipe and the revolution speed of the secondary screw conveyor is controlled in response to pressure values received from the pressure sensor.

All the above solutions suffer from the disadvantage that back blow is detected after its occurrence, that is, these solutions cannot be used to prevent blow-back from occurring.

### OBJECT OF THE INVENTION

It is a first object of the invention to provide a system that reduces the risk of blow-back when feeding biomass material to a pressurized zone.

It is a second object of the invention to provide a method that reduces the risk of blow-back when feeding biomass material to a pressurized zone.

### SUMMARY OF THE INVENTION

The first object of the invention is achieved with a system for transporting biomass material according to claim 1. The system comprises a feeding device comprising a channel comprising an inlet and an outlet for said biomass material and a feed screw arranged at least partly within the channel. The feed screw comprises a screw flight for conveying the biomass material in a biomass transport direction from the inlet to the outlet. The screw flight is also adapted to compress the biomass material during transport to form a gas impermeable plug of biomass material within the channel. A gas impermeable plug is in this context an effectively sealed plug of biomass that serves the purpose of preventing blow back from occurring. However, minor amounts of gas will always be able to penetrate the plug. The screw flight extends from a first end to a second end in the biomass transport direction. The system further comprises at least one primary measuring unit connected to said feeding device between the first end of the screw flight and the outlet. The primary measuring unit is adapted to measure a primary variable, which primary variable is indicative of the gas permeability of the plug. A variable that is indicative of the gas permeability of the plug is a variable that changes in relation to changes in the gas permeability of the plug and can be used, alone or in combination with other data, to determine the gas permeability of the plug. The system further comprises a control unit adapted to continuously receive primary variable values from the primary measuring unit and use said primary variable values to monitor the gas permeability of the plug.

The control unit detects primary variable fluctuations and this makes it possible to identify an increased risk of blow back before blow back occurs. This in turn makes it possible to prevent said blow back from occurring.

Compression of the plug of biomass increases the density of the plug and reduces its porosity and gas permeability. That is, there is a known and inverse relationship between the density of the plug and the gas permeability of the plug, such that an increase of the density of the plug reduces the gas permeability of the plug, and a reduction of the density of the plug increases the gas permeability of the plug. Furthermore, the biomass material is essentially isotropic, that is, the properties of the biomass material are the same in all directions. This means that there exists a known relation between the axial force (along a longitudinal axis of the channel) applied to the plug of biomass and the radial force (orthogonal to the longitudinal axis of the channel) exerted by the plug on the inner surface of the channel. There also exists a known relation between the axial force applied to the plug and the density of the plug. In other words, the gas permeability of the plug can be indirectly monitored by monitoring anyone of a plurality of variables, such as the density of the plug, the radial force exerted by the plug and the radial pressure exerted by the plug. Thus, it is not necessary to determine the gas permeability of the plug and compare it to a gas permeability threshold when monitoring the gas permeability of the plug, as it is possible to determine other variables and compare them to thresholds corresponding to a gas permeability threshold. These thresholds may be experimentally determined and stored in a database to which the control unit has access. It should be noted that the relationship between plug density and gas permeability is independent of channel dimensions, which makes it possible to create a relative small database, wherein is stored thresholds for each combination of biomass material type and pressure within the pressurized zone.

It follows from the above that various types of force and/or pressure measuring units are suitable for use as primary measuring units. Such a force and/or pressure measuring unit may, for example, be adapted to measure a radial force and/or pressure applied by the plug.

Another variable indicative of the gas permeability of the plug is the temperature within the channel, as an increased flow of gas through the plug raises the temperature within the channel. Consequently, various types of temperature measuring units adapted to measure the temperature within the channel are also suitable for use as primary measuring units.

Advantageously, the control unit is adapted to provide a warning signal when the gas permeability of the plug rises above an upper gas permeability threshold, indicating that there is an increased risk of blow back. Suitable types of warning signals are light, sound, text messages etc.

Advantageously, the control unit is adapted to automatically control at least one density regulating means to increase the density of the plug when the gas permeability of the plug is above the upper gas permeability threshold, i.e. when the risk of blow back occurring has increased. This arrangement further reduces the risk of blow back, because the density of the plug is immediately increased when there is an increased risk of blow back occurring. Suitable examples of adjustable density regulating means are the feed screw, a blow back damper arranged at the outlet of the channel, and a feed screw adapted to feed biomass into the channel. The control unit may, for example, be adapted to increase the density of the plug by means of a motor arranged to drive the feed screw or the additional feed screw, or a hydraulic or pneumatic system arranged to move the blow back damper towards the outlet of the channel, when the gas permeability rises above the upper gas permeability threshold.

Another problem associated with plug screw feeders is that the plug may become too densely packed and this may cause plugging of the plug screw feeder, i.e. the radial pressure or the radial force exerted by the plug on the inner surface of the channel causes the plug to become stuck in the channel, which brings the entire process to a halt, increases production costs and increases wear on components. To avoid this, the control unit may be adapted to monitor the radial pressure applied by the plug. This is done by measuring a primary variable that is indicative of the radial pressure exerted by the plug. This solution also makes it possible to optimize the operating conditions of the feeding device, and thus to optimize energy consumption and reduce wear on components. For example, an adequately but not excessively packed plug creates less friction between the plug and the inner surface of the channel, which makes it easier to convey the biomass material through the channel, reduces power consumption of the system and reduces wear on the components. It is not necessary to measure or determine the radial pressure to monitor the radial pressure, the radial pressure can be indirectly monitored by measuring and monitoring other variables indicative of the radial pressure applied by the plug, e.g. a radial force exerted by the plug. A variable that is indicative of the radial pressure exerted by the plug is a variable that changes in relation to changes in the radial pressure exerted by the plug and can be used, alone or in combination with other data, to determine the radial pressure exerted by the plug.

Advantageously, the control unit is adapted to provide a warning signal when the radial pressure of the plug is above an upper radial pressure threshold, indicating that the plug is too densely packed, to alert a user of the system to the fact that there is an increased risk of plugging. Suitable types of warning signals are light, sound, text messages etc.

Advantageously, the control unit is adapted to automatically control at least one density regulating means to reduce the density of the plug when the radial pressure applied by the plug is above the upper radial pressure threshold. This arrangement reduces response times and thus the risk of plugging.

The radial pressure exerted by the plug, e.g. on an inner surface of the channel or a measuring unit arranged within the channel, has two components, namely the radial pressure exerted by the biomass material that constitutes the plug and the pressure exerted by the gas within the pores of the plug. The radial pressure exerted by the biomass material usually exceeds the pressure exerted by the gas with a magnitude such that the total radial pressure can be used to monitor the gas permeability of the plug. However, it may still be advantageous to measure the force and/or pressure applied by the plug where the density is highest, to ensure that the difference between the radial pressure exerted by the biomass material and the gas pressure is as large as possible.

The primary measuring unit is arranged to measure the primary variable between the first end of the screw flight and the outlet of the channel. This arrangement makes it possible to measure a variable associated with the plug and thus to monitor the gas permeability of the plug. The exact location of this position depends on several variables. For example, the density of the plug is usually highest near the second end of the screw flight. Therefore, it is advantageous to arrange said primary measuring unit within a distance from the second end having a length of 10 times the diameter of the channel at said second end, preferably 5 times the diameter of the channel at said second end, and even more preferably 1 times the diameter of the channel at said second end.

Advantageously, at least one primary measuring unit is arranged to come into contact with the plug of biomass. For example, it may be arranged, completely or partially, within the channel. However, it is also possible to arrange at least one primary measuring unit so that it does not come into contact with the plug of biomass. For example, it may be attached to the outside of the housing that defines the channel.

The primary measuring unit according to the invention may be of any suitable type that can be used to measure any variable that can be used to monitor the gas permeability of the plug of biomass. For example, the primary measuring unit may be a pressure measuring unit, such as a pressure sensor or a pressure transducer (e.g. of membrane type). A pressure measuring unit may, for example, be arranged to be in contact with the plug of biomass, and be adapted to measure the radial pressure applied to the pressure measuring unit. Alternatively, the primary measuring unit may be a force measuring unit, e.g. a load cell or a force transducer. A force measuring unit may, for example, be arranged within the parting plane between two halves of a housing that defines the channel. The primary measuring unit may also comprise a strain gauge arranged to be deformed when the radial pressure exerted by the plug increases. A strain gauge may, for example, be attached to the outside of the housing that defines the channel. Another suitable type of measuring unit is an accelerometer, that measures vibrations on the surface of the housing. It is also possible to use more than one primary measuring unit and the above-mentioned measuring units may be combined in many ways. It is, for example, suitable to combine a force and/or pressure measuring unit with a temperature measuring unit, such as a temperature sensor adapted to measure the temperature within the channel.

The system may comprise more than one primary measuring unit adapted to measure the same or different variables. An additional measuring unit may, for example, be attached to a blow back damper arranged to apply a counter pressure on the plug of biomass within the channel of the feeding device.

A gas pressure measuring unit may be arranged to continuously measure the gas pressure within a pressurized zone connected to the outlet of the channel, and the control unit may be adapted to continuously receive gas pressure values from the gas pressure measuring unit and use said gas pressure values to determine the upper gas permeability threshold, so that the upper gas permeability threshold is lowered when the gas pressure within the pressurized zone increases, and is increased when the gas pressure within the pressurized zone is reduced. This embodiment is advantageous in that it takes into account the fact that an increase of the gas pressure within the pressurized zone increases the risk of blow back. It should be noted that the relationship between plug density and gas permeability is independent of channel dimensions, which makes it possible to create a relatively simple and small database, wherein may be stored different types of threshold values for each combination of biomass material type and pressure within the pressurized zone. This significantly reduces the amount of work required to set suitable variables for the system.

The second object is achieved with a method for preventing blow back in a system for transporting biomass material as described in independent claim 11. The system comprises a feeding device comprising a channel comprising an inlet and an outlet for said biomass material, and a feed screw arranged at least partly within the channel and comprising a screw flight for conveying the biomass material in a biomass transport direction from the inlet to the outlet. The screw flight is also adapted to compress the biomass material during transport to form a gas impermeable plug of biomass material within the channel and extends from a first end to a second end in the biomass transport direction. The method comprises the steps of at least one primary measuring unit connected to said feeding device between the first end of the screw flight and the outlet continuously measuring a primary variable indicative of the gas permeability of the plug, said primary measuring unit continuously transmitting primary variable values to a control unit, and said control unit using said primary values to monitor the gas permeability of the plug.

As described above, monitoring the gas permeability of the plug of biomass makes it possible to identify an increased risk of blow back and to take preventive measures before blow back occurs.

Advantageously, the method comprises the step of a primary measuring unit measuring a force and/or pressure exerted by the plug. Alternatively, or in combination, the method may comprise the step of a primary measuring unit measuring a temperature within the channel.

The method advantageously comprises the step of said control unit providing a warning signal (sound, text, light etc.) when the gas permeability of the plug is above an upper gas permeability threshold. This step makes it possible for an operator to reduce the gas permeability of the plug before blow back occurs.

Alternatively, or in combination, the method may comprise the step of said control unit automatically controlling at least one density regulating means to increase the density and reduce the gas permeability of the plug when the gas permeability of the plug is above the upper gas permeability threshold. This step further reduces the risk of blow back.

The method may comprise the step of said control unit using primary variable values indicative of the radial pressure exerted by the plug to monitor the radial pressure exerted by the plug.

Advantageously, the method comprises the step of said control unit providing a warning signal (sound, light etc.) when the radial pressure is above an upper radial pressure threshold, i.e. when there is an increased risk of plugging. This step makes it possible for an operator to adjust the density of the plug before plugging occurs.

Advantageously, the method comprises the step of said control unit automatically controlling at least one density regulating means to reduce the density of the plug when the radial pressure of the plug exceeds the upper radial pressure threshold. This step further reduces the risk of plugging.

The method may also comprise the step of measuring said primary variable within a distance from the second end of the screw flight having a length of 10 times the diameter of the channel at said second end, preferably 5 times the diameter of the channel at said second end, and even more preferably 1 times the diameter of the channel at said second end.

The method may also comprise the steps of a gas pressure measuring unit continuously measuring the gas pressure within a pressurized zone connected to the outlet of the channel, said gas pressure measuring unit continuously transmitting gas pressure values to the control unit, and said control unit using said gas pressure values to determine the upper gas permeability threshold. This embodiment makes it possible to optimize the operating conditions of the feeding device.

The control unit according to the invention may be adapted to perform many different functions. The control unit may comprise any suitable number of control means, each adapted to perform one or more of these functions. These control means may be arranged together or at a distance from one another.

The plug of biomass is created due to friction between the biomass and the inner surface of the channel accommodating the feed screw. In prior art devices, the channel and the feed screw have excessive lengths to ensure that the plug of biomass becomes gas impermeable. More precise control of the gas permeability of the plug means that the channel and the feed screw can be shortened, in comparison to prior art arrangements, with shorter response times as a result. This is especially the case when a blow back damper is used to create the plug of biomass.

The system according to the invention can be used to transport any suitable type of biomass material, e.g. wood chips, straw, cane, bagasse etc.

### BRIEF DESCRIPTION OF THE DRAWINGS

The present invention will be further explained hereinafter by means of non-limiting examples and with reference to the appended drawing, wherein:

FIG. 1 shows a schematic view of a first embodiment of a system according to the invention; and

FIG. 2 shows a schematic view of a second embodiment of a system according to the invention.

### DETAILED DESCRIPTION OF THE INVENTION

In the following description, like parts are referred to an indicated by like reference signs. Some parts have been removed from the figures for the sake of clarity.

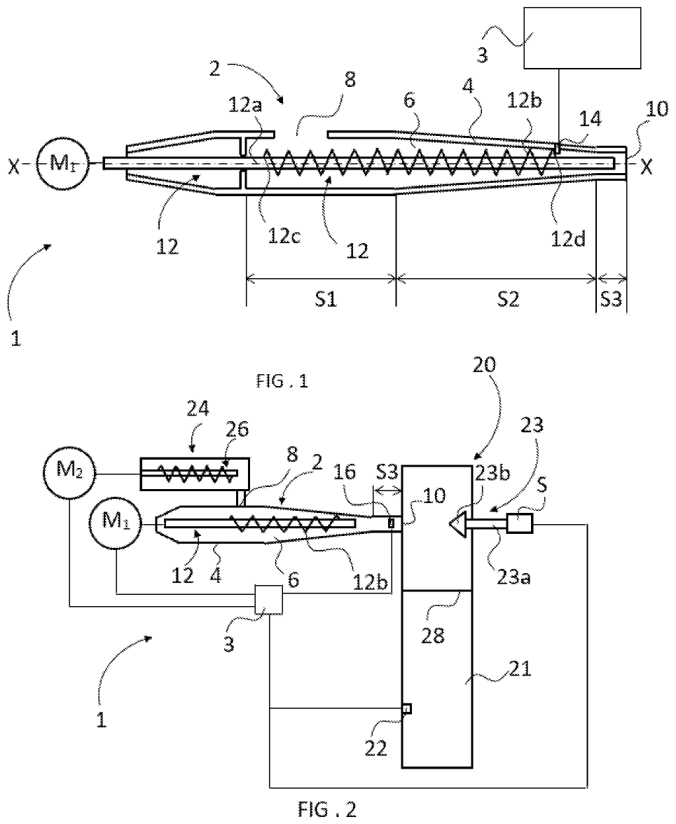

FIG. 1 illustrates a system 1 according to a first embodiment of the invention comprising a feeding device 2 in the form of a plug screw feeder and a control unit 3.

The feeding device 2 comprises a housing 4 that defines a channel 6 that extends along a longitudinal axis X of the feeding device 2. The channel 6 is divided into an inlet section 51, an intermediate section S2 and an outlet section S3. The inlet section 51 comprises an inlet 8 for biomass material and the outlet section S3 comprises an outlet 10 for biomass material. The inlet 8 is adapted to be connected to a feeding device (not shown) for feeding 25 biomass into the channel 6. Such a feeding device may, for example, comprise a force feed screw adapted to transport the biomass towards the inlet 8. The outlet 10 is adapted to be connected to a charger (not shown) to allow the biomass to be discharged from the channel 6 and into said charger.

A feed screw 12 extends into the housing 4 along the longitudinal axis X of the feeding device 2 towards the outlet 10. The feed screw 12 is adapted to convey the biomass material in a biomass transport direction from the inlet 8 to the outlet 10. The feed screw 12 comprises a central shaft 12a, which at one end is connected to and arranged to be rotated by a first drive mechanism M₁. A screw flight 12b in the form of a screw helix (schematically shown) adapted to convey the biomass in the biomass transport direction extends around a portion of the central shaft 12a, from a first end 12c to a second end 12d, with a suitable pitch. The second end 12d is located at a distance from the outlet 10.

The biomass is compressed during transport through the channel 6, so that a gas impermeable plug of biomass is formed within the channel 6. Most of this compression occurs within the intermediate section S2, which has a narrowing cross-section towards the outlet 10, unlike the inlet and outlet sections S1 and S2, which have essentially constant cross-sections along the longitudinal axis X of the feeding device 2.

The biomass may be subjected to additional treatment during transport through the channel 6. The biomass may, for example, be subjected to dewatering, in which case additional pipes (not shown) may be connected to the channel 6 for transporting excess fluids away from the channel 6. Such means are known to the skilled person and will not be described in detail herein.

A primary measuring unit 14 in the form of a pressure sensor extends into the channel 6 at the second end 12d of the screw flight 12b. The pressure sensor is connected to the control unit 3. The pressure sensor is in this embodiment arranged to come into contact with the plug within the channel 6. The pressure sensor is adapted to measure a primary variable, in this embodiment the radial pressure (orthogonal to the longitudinal axis X of the feeding device 2) applied thereto. This radial pressure has two components, the pressure applied by the biomass that constitutes the plug and the pressure applied by the gas present within the pores of the plug. The pressure exerted by the gas is usually more or less negligible in comparison to the radial pressure exerted by the biomass. However, it is still advantageous to position the pressure sensor at the second end 12d of the screw flight 12b, because this is where the density of the plug is highest and thus also where the difference between the radial pressure applied by the biomass and the pressure applied by the gas is the largest. Thus, by positioning the pressure sensor at the second end 12d of the screw flight 12b is ensured that the radial pressure applied by the gas is negligible in comparison to the radial pressure applied by the biomass, and it can be assumed that the measured radial pressure is equal to the radial pressure exerted by the plug of biomass.

The measured radial pressure is indicative of the gas permeability of the plug, i.e. it can be used to determine the gas permeability of the plug. The control unit 3 compares the primary values, i.e. radial pressure values, received from the pressure sensor 14 to a lower radial pressure threshold corresponding to an upper gas permeability threshold to determine if the there is an increased risk of blow back. If the comparison shows that the radial pressure has dropped below the lower radial pressure threshold, then the control unit 3 sends out an alert (e.g. in the form of a light, sound or text message) to make an operator of the system aware that there is an increased risk of blow back. The operator may then, for example, reduce the rotational speed of the motor M₁ to increase the density of the plug and make it essentially gas impermeable.

The control unit 3 may also be adapted to compare the primary values to an upper radial pressure threshold to determine if there is an increased risk of plugging of the channel 6. If the comparison reveals that the density of the plug is so high that there is an increased risk of plugging, then the control unit 3 sends out an alert to make the operator aware that there is an increased risk of plugging. The operator may then, for example, increase the rotational speed of the motor M₁ to reduce the density of the plug and thus the risk of plugging.

FIG. 2 is a schematic view of a system 1 according to a second embodiment of the invention. The system 1 comprises a feeding device 2 similar to the feeding device 2 in FIG. 1, the only difference being that the primary measuring unit 16 in FIG. 2 is a strain gauge (schematically shown) attached to an outside of the housing 4 of the feeding device 2. The electric resistance of the primary measuring unit 16 varies with the length of the primary measuring unit 16 and thus the length of the corresponding part of the housing 4, and the length of the corresponding part of the housing 4 varies with the radial pressure that the plug of biomass exerts on the inside of the channel 6, wherefore the electric resistance is a variable that is indicative of the radial force applied by the plug of biomass and thus also the gas permeability of the plug of biomass. The primary measuring unit 16 in FIG. 2 is arranged within the outlet section S3 close to the outlet 10.

The system 1 also comprises a feeding device 24 for delivering biomass to the feeding device 2, which feeding device 24 comprises a feed screw 26 driven by a second drive mechanism M₂.

The outlet 10 of the feeding device 2 is connected to a charger 20 arranged to receive biomass from the feeding device 2. A blow back damper 23 extends into the charger 20. The blow back damper 23 is arranged to be moved reciprocally towards and away from the outlet 10 of the feeding channel 6 by means of a hydraulic or pneumatic system S. The blow back damper 23 comprises a shaft 23a and a damper head 23b, which damper head 23b is moveable between a first position, in which it closes the outlet 10, and a second position, in which the damper head 23b is sufficiently far removed from the outlet 10 to ensure that the damper head 23b does not interact with the biomass being discharged through the outlet 10. The damper head 23b may occupy any position between the first and second positions, and is during use often positioned at a distance from the outlet 10 but still within reach of the biomass that is discharged through the outlet 10, so that the damper head 23b is used to shred the plug of biomass being discharged from the feeding device 2 while exerting a counter-pressure on said plug of biomass.

The biomass may be subjected to further treatment within the charger 20 and for this purpose, additional means (now shown), e.g. pipes, may be arranged within or connected to the charger. Such means are known to the skilled person and will not be described in detail herein.

The charger further comprises a charger outlet 28, through which biomass is conveyed to a pressurized reactor 21, wherein the biomass may be subjected to different types of treatments. A gas pressure measuring unit 22 in the form of a gas pressure sensor is arranged within the charger 21 and adapted to continuously measure the gas pressure within the charger 21 and send measured gas pressure values to the control unit 3.

The control unit 3 is connected to both the strain gauge and the gas pressure measuring unit 22, the hydraulic or pneumatic system S as well as to the first and second drive mechanisms M₁ and M₂, so that the control unit 3 may control the hydraulic or pneumatic system S and the first and second drive mechanisms M₁, M₂ in response to data received from the primary measuring unit 16 and the gas pressure measuring units 22.

The method for preventing blow back through the feeding device 2 will now be described in detail with reference to FIG. 2.

The biomass is conveyed through the feeding device 24 by means of the feed screw 26 driven by the second drive mechanism M₂. The biomass is delivered through the inlet 8 into the channel 6 within the feeding device 2. The first drive mechanism M₁ rotates the feed screw 12 and the screw flight 12b extending along a portion of the feed screw 12 conveys the biomass in the biomass transport direction towards the outlet 10. During transport the biomass is compressed, partly due to the narrowing cross-section of the channel 6, and forms an essentially gas impermeable plug within the channel 6.

The blow back damper head 23b is initially positioned in the first position, wherein the damper head 23b closes the outlet 10. Thus, the damper head 23b prevents the biomass from entering the chamber and prevents gas from the pressurized reactor from 21 entering the feeding device 2. The damper head 23b applies a counter pressure to the biomass within the channel 6 and contributes to the formation of the gas impermeably plug.

The compressed biomass within the channel 6 exerts an increasing pressure on the damper head 23b and eventually pushes the damper head 23b in a direction away from the outlet 10, so that the plug of biomass may be discharged through the outlet 10 and into the charger 23, wherein it is shredded by the damper head 23b and the biomass falls towards the bottom of the charger 23. The counter pressure applied by the blow back damper 23 is selected so that the plug of biomass formed within the channel 6 is essentially gas impermeable when the damper head 23b is moved to an intermediate position.

The primary measuring unit 16 is adapted to measure a primary variable, in this case the electric resistance of the strain gauge, which is indicative of the gas permeability of the plug of biomass, and transmit primary variable values to the control unit 3. The control unit 3 may then use these received primary variable values to determine the gas permeability of the plug of biomass. This makes it possible for the control unit 3 to ensure that the plug of biomass is sufficiently dense and essentially gas impermeable when the damper head 23b is pushed back. The control unit 3 may, for example, be adapted to control the hydraulic or pneumatic system S to prevent the damper head 23b from being pushed back until a comparison between the determined gas permeability of the plug and an upper gas permeability threshold shows that the plug is essentially gas impermeable.

The density of the plug of biomass may vary over time, e.g. due to a change in the flow of biomass through the channel 6, and such changes may increase the risk of blow back. The main purpose of the primary measuring unit 16 is to prevent this from happening. As explained above, the electric resistance of the primary measuring unit 16 changes with the pressure applied by the plug to the inside of the housing 4, and can thus be used to determine the gas permeability of the plug. The control unit 3, which continuously receives data from primary measuring unit 16, uses these primary variable values to determine the gas permeability of the plug. If the gas permeability rises above the upper gas permeability threshold, then the control unit 3 acts to ensure that the density is increased. The control unit 3 may, for example, increase the rotational speed of the second drive mechanism M₂ to increase the rotational speed of the feed screw 26 and thus increase the flow of biomass into the feeding device 2. The control unit 3 may also, or alternatively, decrease the rotational speed of the first drive mechanism M₁ to decrease the rotational speed of the feed screw 12 and thus increase the pressure the plug of biomass exerts on the housing 4. Finally, the control unit 3 may regulate the hydraulic or pneumatic system S, so that the counter pressure applied by the blow back damper 23 is increased, which also moves the damper head 23b in a direction towards the outlet 10.

A change in gas pressure within the reactor 21 unit increases the risk of blow back. Therefore, the gas pressure measuring unit 22 is adapted to measure the gas pressure within the reactor 21. The control unit 3 continuously receives gas pressure values from the gas pressure measuring unit 22 and uses them to determine an optimal value for the upper gas permeability threshold, so that the upper gas permeability threshold is lowered when the gas pressure within the reactor 21 increases, and is raised when the gas pressure within the reactor 21 is reduced.

As for the system shown in FIG. 1, the control unit 3 may also be adapted to compare the radial pressure applied by the plug of biomass with a predetermined upper radial pressure threshold, to ensure that the channel 6 does not become plugged. An increase of the radial pressure above the upper radial pressure threshold would in this embodiment cause the control unit to regulate one or more of hydraulic or pneumatic system S and the first and second drive mechanisms M₁, M₂ to reduce the density of the plug.

Of course, the upper and lower thresholds should be selected so that the control unit acts before blow back occurs, and/or before the channel becomes plugged.

The scope of protection is not limited by the above described embodiments and features from different embodiments may be combined in many ways. For example, the primary measuring units in FIGS. 1 and 2 may any suitable type of primary measuring units and the control unit in FIG. 2 may be adapted to provide a warning signal when the density of the plug exceeds the upper threshold or falls below the lower threshold.

1. A system for transporting biomass material, which system comprises a feeding device comprising:
a channel comprising an inlet and an outlet for said biomass material; and
a feed screw arranged at least partly within the channel and comprising a screw flight for conveying the biomass material in a biomass transport direction from the inlet to the outlet, which screw flight is also adapted to compress the biomass material during transport to form a gas impermeable plug of biomass material within the channel, and which screw flight extends from a first end to a second end in the biomass transport direction;
characterized in that said system further comprises:
at least one primary measuring unit adapted to continuously measure a primary variable indicative of the gas permeability of the plug, which primary measuring unit is connected to said feeding device between the first end of the screw flight and the outlet; and
a control unit adapted to continuously receive primary variable values from the primary measuring unit and use said primary variable values to monitor the gas permeability of the plug.

2. A system according to claim 1, which system comprises a primary measuring unit adapted to measure a force and/or pressure exerted by the plug.

3. A system according to claim 1, which system comprises a primary measuring unit adapted to measure a temperature within the channel.

4. A system according to claim 1, wherein said control unit is adapted to provide a warning signal when the gas permeability of the plug is above an upper gas permeability threshold.

5. A system according to claim 4, wherein said control unit is adapted to automatically control at least one density regulating means to increase the density of the plug when the gas permeability of the plug is above the upper gas permeability threshold.

6. A system according to claim 1, wherein said primary variable is indicative of the radial pressure exerted by the plug and the control unit is adapted to use said primary variable values to monitor the radial pressure exerted by the plug.

7. A system according to claim 6, wherein said control unit is adapted to provide a warning signal when the radial pressure is above an upper radial pressure threshold.

8. A system according to claim 7, wherein said control unit is adapted to automatically control at least one density regulating means to reduce the density of the plug when the radial pressure of the plug exceeds the upper radial pressure threshold.

9. A system according to claim 1, wherein said primary measuring unit is arranged within a distance from the second end of the screw flight, which distance is 10 times the diameter of the channel at said second end, preferably 5 times the diameter of the channel at said second end, and even more preferably 1 times the diameter of the channel at said second end.

10. A system according to claim 4, which system comprises a gas pressure measuring unit arranged to continuously measure the gas pressure within a pressurized zone connected to the outlet, and wherein said control unit is adapted to continuously receive gas pressure values from the gas measuring unit and use said gas pressure values to determine the upper gas permeability threshold.

11. A method for preventing blow back in a system for transporting biomass material, which system comprises a feeding device comprising:
a channel comprising an inlet and an outlet for said biomass material; and
a feed screw arranged at least partly within the channel and comprising a screw flight for conveying the biomass material in a biomass transport direction from the inlet to the outlet, which screw flight is also adapted to compress the biomass material during transport to form a gas impermeable plug of biomass material within the channel, and which screw flight extends from a first end to a second end in the biomass transport direction;
which method is characterized in that it comprises the steps of:
at least one primary measuring unit connected to said feeding device between the first end of the screw flight and the outlet continuously measuring a primary variable indicative of the gas permeability of the plug;
said primary measuring unit continuously transmitting primary variable values to a control unit; and
said control unit using said primary values to monitor the gas permeability of the plug.

12. Method according to claim 11, which method comprises the step of a primary measuring unit measuring a force and/or pressure exerted by the plug.

13. Method according to claim 11, which method comprises the step of a primary measuring unit measuring a temperature within the channel.

14. A method according to claim 11, which method comprises the step of said control unit providing a warning signal when the gas permeability of the plug is above an upper gas permeability threshold.

15. A method according to claim 14, which method comprises the step of said control unit automatically controlling at least one density regulating means to increase the density of the plug when the gas permeability of the plug is above the upper gas permeability threshold.

16. A method according to claim 11, wherein the primary variable is indicative of the radial pressure exerted by the plug, which method comprises the step of said control unit using said primary variable values to monitor the radial pressure exerted by the plug.

17. A method according to claim 16, which method comprises the step of said control unit providing a warning signal when the radial pressure is above an upper radial pressure threshold.

18. A method according to claim 17, which method comprises the step of said control unit automatically controlling at least one density regulating means to reduce the density of plug when the radial pressure of the plug exceeds the upper radial pressure threshold.

19. A method according to claim 11, which method comprises the step of measuring said primary variable within a distance from the second end of the screw flight, which distance is 10 times the diameter of the channel at said second end, preferably 5 times the diameter of the channel at said second end, and even more preferably 1 times the diameter of the channel at said second end.

20. A method according to claim 14, which method comprises the steps of:
a gas pressure measuring unit continuously measuring the gas pressure within a pressurized zone connected to the outlet;
said gas pressure measuring unit continuously transmitting gas pressure values to the control unit; and
said control unit using said gas pressure values to determine the upper gas permeability threshold.
